(19) United States
(12) Patent Application Publication
(10) Pub. No.: US 2026/0335265 A1
(43) Pub. Date: Oct. 29, 2026

(54) ANTICANCER COMPOSITION COMPRISING MALATE METAL SALT

(71) Applicant: BIOSOLYX, Daegu (KR)

(72) Inventor: Keun-Yeong JEONG, Seoul (KR)

(21) Appl. No.: 19/114,503

(22) PCT Filed: Sep. 27, 2022
(86) PCT No.: PCT/KR2022/014491

§ 371 Date: Mar. 24, 2025

(51) Int. Cl. A61K 31/194; A61K 31/19; A61K 45/06; A61P 35/00; A61P 37/04

(52) U.S. Cl. CPC A61K 31/194; A61K 31/19; A61K 45/06; A61P 35/00; A61P 37/04

(57) ABSTRACT

The present invention relates to anticancer uses of a malate metal salt. Particularly, the present invention relates to a pharmaceutical composition for preventing or treating cancer and an anticancer adjuvant pharmaceutical composition, the compositions comprising a malate metal salt as an active ingredient. The composition for preventing or treating cancer, comprising a malate metal salt as an active ingredient, of the present invention has excellent anticancer effects by exhausting NAD⁺, which is an energy source in cancer cells, and increasing immunoactivity, increases insulin secretion through an increase in malic acid activity, and thus solves problems of decreased insulin secretion or resistance induced by toxicity from various anticancer treatments. In addition, when co-administered during the administration of other anticancer drugs or anticancer radiotherapy, the present invention increases a responsiveness to the anticancer drugs and radiotherapy and thus also has very excellent effects as an anticancer adjuvant.

### TECHNICAL FIELD

The present invention relates to anticancer uses of a malate metal salt. Particularly, the present invention relates to a pharmaceutical composition for preventing or treating cancer and an anticancer adjuvant pharmaceutical composition comprising a malate metal salt as an active ingredient.

### BACKGROUND ART

Malate is basic metabolite of TCA cycle (Tricarboxylic Acid Cycle) and are derived from succinate. However, as cancer cells grow in vivo, the microenvironment within tumor tissues becomes in a hypoxic state, and in hypoxic cancer cells, the TCA cycle is significantly altered, and succinate and fumarate are accumulated. In other words, the relative abundance of several TCA cycle intermediates is altered in hypoxic cancer cells, and changes in the expression of various enzymes involved in the TCA cycle are also observed.

Reduced levels of malate in cancer cells due to altered utilization of intermediates derived from the TCA cycle leads to decreased activity of malate dehydrogenase 2 (MDH2), and hypoxic cancer cells primarily rely on glutamine-utilizing biochemical operations to compensate. Specifically, cancer cells in hypoxic environments induce an increase of aspartate in cytosolic by increasingly supply glutamine within cells. Increased aspartate activates aspartate aminotransferase to be converted to oxaloacetate, reduces oxaloacetate to malate if the level of oxaloacetate becomes increased in the cytosolic, and highly dependent on malate dehydrogenase 1 (MDH1), which and oxidizes NADH to NAD⁺. As a result, the activation of MDH1 creates a tumor microenvironment favorable for cancer cells survival by providing NAD⁺, which is used as a continuous energy source in cancer cells.

In the meanwhile, cancer and diabetes are known to be closely related. Insulin is a hormone that affects the growth process of cells, and when the pancreas's ability to secrete insulin is impaired, sugar builds up in the blood, leading to diabetes. Patients with diabetes maintain high blood sugar levels due to poor insulin regulation, which leads to ongoing damage to DNA, which can lead to cancer. On the other hand, cancer rapidly depletes energy during its growth phase, which impairs insulin regulation, and the damaged insulin-secreting cells bring about cancerous diabetes. Like this, cancer and diabetes are inextricably linked, working in both directions, and there has been a need for a treatment. As malic enzymes are known to play an important role in the secretion of insulin, it is considered that modulating the activity of malic enzymes capable of solving the problems such as insulin secretion or resistance induced by the toxicity of various cancer treatments, but no drug has been developed that can effectively regulate malic acid to treat cancerous diabetes until now.

Based on the above background, the inventors hypothesized that depleting NAD⁺, an energy source that is supplied in excess to cancer cells in hypoxic environments, could effectively kill cancer cells. Previously, the accumulation of intermediates such as succinate and fumarate due to changes of the TCA cycle was known to promote the growth and differentiation of cancers in hypoxic environments (Nature Communications, 2020, 11: 102), but the inventors sought to deplete NAD⁺ by reducing NAD⁺ to NADH, and instead supply malate as a metal salt to the cells to accumulate intracellular malate. As a result, the above malate metal salt treatment increased insulin secretion by activating malic acid enzymes, induced the activity of MDH2 and malic acid enzymes in cancer cells to restore the TCA cycle, and depleted the energy source NAD⁺, thus confirming that malate metal salts have excellent anticancer effects.

### DISCLOSURE OF THE INVENTION

### Technical Problem

It is therefore an object of the present invention to provide a pharmaceutical composition for preventing or treating cancer comprising a malate metal salt as an active ingredient.

It is another object of the present invention to provide an anticancer adjuvant pharmaceutical composition comprising the malate metal salt as the active ingredient for enhancing a response to a second cancer drug or an anticancer radiotherapy.

It is still a further object of the present invention to provide a method of preventing or treating cancer comprising the step of administering the malate metal salt to an individual in need thereof.

It is still a further object of the present invention to provide a use of the malate metal salt for preventing or treating cancer or cancerous diabetes.

It is still a further object of the present invention to provide a use of the malate metal salt for a use in a preparation of a medicament for preventing or treating cancer or cancerous diabetes.

### Technical Solution

The present invention will now be described more fully hereinafter with reference to the accompanying drawings. Meanwhile, each of the descriptions and embodiments disclosed in the present invention can also be applied to other descriptions and embodiments. That is, all combinations of the various elements disclosed in the present invention fall within the scope of the invention. Furthermore, the scope of the invention is not to be considered limited by the specific descriptions set forth below.

One aspect of the present invention to accomplish the above object provides a pharmaceutical composition for preventing or treating cancer comprising a malate metal salt as an active ingredient.

In the present invention, the term “malate metal salt” refers to a compound that can be produced or synthesized in the form of a metal ion bound to a malate. Due to the nature of anticancer drugs, which may be administered frequently and in large doses, metal components that cannot be readily metabolized in the body are excluded from the scope of the present invention. The inventor anticipated that the treatment of cancer cells with malate metal salts would increase the activity of malate dehydrogenase 2 (MDH2) and malic enzymes such as malic enzyme 2 (ME2) and malic enzyme 3 (ME3), thereby killing cancer cells by exhausting NAD⁺. In one specific embodiment of the present invention, the treatment of various cancer cell lines with calcium malate or calcium di-malate resulted in a significant increase in the activities of MDH2, ME2, and ME3 compared to untreated control groups (See FIG. 1), and surprisingly, a dramatic exhaustion of NAD⁺ in cancer cells (See FIG. 2), resulting in the death of cancer cells (See FIGS. 4, 5, 10, and 11). Accordingly, a pharmaceutical composition comprising a malate metal salt of the present invention can be very useful for preventing or treating cancer.

In the present invention, “malate metal salt” is used as a means of increasing the concentration of malate in cancer cells by dissociating the malate when administered into the cancer cells, and is not particularly limited as to its type and the ratio of malate to metal ion, as long as it can accomplish this purpose. For example, the malate metal salt may be calcium malate, calcium dimalate, zinc malate, magnesium malate, sodium malate, potassium malate, iron malate, chromium malate, copper malate, manganese malate, and the like.

In one embodiment of the present invention, the pharmaceutical composition may further comprise DCA (Dichloroacetic acid) as an active ingredient in addition to a malate metal salt. The DCA, also named dichloroacetic acid, is an acetic acid analogue in which two of the three hydrogen atoms of the methyl group of acetic acid are replaced by chlorine atoms. Since the DCA activates pyruvate dehydrogenase, the inventors anticipated that pyruvate, which is increased by MDH2 or malic enzymes within cancer cells, could actively participate in the repair of the TCA cycle by the DCA treatment. In one specific embodiment of the present invention, the treatment of various kinds of cancer cells with calcium malate and DCA was found to increase the activity of MDH2 and malic enzymes to a higher level than the treatment with the metal salt malate (FIGS. 1 and 2) and to effectively kill cancer cells (FIGS. 6 and 12). Therefore, the DCA may be further comprised to further enhance the anticancer effect of the pharmaceutical composition according to the present invention.

As used herein, the term “cancer” refers to a disease involving the death regulation of cells, which is caused by the hyper-proliferation of cells when the normal balance of cell death is disturbed. In the present invention, the cancer includes both malignant tumors and benign tumors, and can be, for example, solid tumors such as liver cancer, lung cancer, colon cancer, brain cancer, kidney cancer, pancreatic cancer, and breast cancer, and can be, for example, cancerous diabetes, but the types of the cancer of the present invention are not limited by the above examples. The composition for preventing or treating cancer according to the present invention have a therapeutic effect on any cancer that can utilize intracellular NAD⁺ as an energy source.

As used in the present invention, the term “treatment” refers to intervening to alter the natural processes of an individual or cells having a disease, which may be performed during the progression of a pathologic condition or to prevent it. Desired therapeutic effects include preventing the occurrence or recurrence of a disease, relieving symptoms, reducing any direct or indirect pathologic consequences of the disease, preventing metastasis, reducing the rate of disease progression, alleviating or temporarily relieving a disease condition, causing remission, or improving prognosis. In particular, the present invention includes any act of ameliorating the course of cancer by administrating a composition comprising a malate metal salt. Also, the term “preventing” refers to any act of inhibiting or delaying the development of cancer by the administration of the composition.

The content of the malate metal salt in the pharmaceutical composition is not particularly limited, but may include an amount of 0.0001 to 2000 mg/kg in general, and more concretely, an amount of 0.001 to 1000 mg/kg.

The pharmaceutical composition of the present invention can be administered to an individual who has developed or is at risk of developing cancer. As used in the present invention, the term “individual” refers to any animal including human beings.

The pharmaceutical composition of the present invention can be administered to a subject in a pharmaceutically effective amount. As used herein, the term “administering” refers to the introduction of a pharmaceutical composition of the invention into a subject by any suitable means, and the route of administration can be various such as oral or parenteral, as long as it can reach the target tissue. Examples of routes of administration include, but are not limited to, oral, intramuscular, intravenous, arterial, subcutaneous, intraperitoneal, pulmonary, and nasal, and may be administered, for example, subcutaneously, orally, or intravenously.

As used in the present invention, the term “pharmaceutically effective amount” means an amount sufficient to prevent and/or treat cancer at a reasonable benefit/risk ratio applicable to the medical use. Appropriate dosage and number of doses may be selected according to manners known in the art, and the amount and the number of doses of pharmaceutical composition of the invention actually administered may be appropriately confirmed by a variety of factors, such as a kind of symptom to be treated, the route of administration, gender, health status, diet, age, weight, and severity of disease of an individual.

The composition comprising the malate metal salt of the present invention as an active ingredient can be formulated not only in a pharmaceutical composition but also in the form of a food composition with functionality to alleviate cancer.

When the composition of the present invention is formulated in the form of a food composition, the food composition may include additional ingredients that are commonly used in food to enhance odor, taste, appearance, and the like. For example, food additives may be added. The food additives are selected and used in appropriate amounts depending on a kind of food.

The food composition may be formulated as a health functional food, wherein the term health functional food is synonymous with food for special health use (FoSHU), which refers to a food with high medical or health benefits that has been processed to efficiently exert biomodulatory functions in addition to providing nutrition. The functional food may be manufactured in various forms, such as tablets, capsules, powders, granules, liquids, pills, etc. in order to achieve useful effects for the amelioration of cancer.

Another aspect of the present invention is an anticancer adjuvant pharmaceutical composition comprising a malate metal salt as an active ingredient to enhance a responsiveness to a second anticancer drug.

Another aspect of the present invention is a pharmaceutical composition for preventing or treating of cancer comprising a malate metal salt and a second anticancer drug as an active ingredient.

The malate metal salt is as described above.

In addition to having an anticancer effect alone, the composition comprising the malate metal salt according to the present invention also have an excellent effect as the adjuvant pharmaceutical composition for the purpose of enhancing the responsiveness to the second anticancer drug. Therefore, the composition comprising the malate metal salt can be used as the pharmaceutical composition for preventing or treating cancer with the second anticancer drug to the malate metal salt as the active ingredient.

In the present invention, the term “second anticancer drug” refers to any drug having anticancer activity other than the malate metal salt of the present invention. The scope of the second anticancer drug in the present invention is not particularly limited, and a person those skilled in the art can select and use the appropriate type according to the type and progression of the cancer for the purpose of curing, controlling, or alleviating symptoms of the cancer. The second anticancer drug may be, for example, but not limited to, a cytotoxic anticancer drug, a targeted anticancer drug, an immune anticancer drug, or a metabolic anticancer drug.

In the present invention, the cytotoxic anticancer drug is a drug that exerts an anticancer effect by attacking cancer cells that divide indiscriminately at a faster rate than normal cells, as that term is commonly used in the art to which the present invention belongs. The cytotoxic anticancer drugs include alkylating drugs, antimetabolites, and natural anticancer drugs.

The alkylating drugs include nitrogen mustards (e.g., cyclophosphamide, chlormethine, uramustine, melphalan, chlorambucil, ifosfamide, bendamustine, etc.), alkyl sulfonates (e.g., busulfan, procarbazine, etc.), nitrosoureas (e.g., carmustine, lomustine, streptozocin, etc.), platinum-based alkylating drugs (e.g., cisplatin, carboplatin, dicycloplatin, eptaplatin, lobaplatin, myriplatin, nedaplatin, oxaliplatin, picoplatin, satraplatin, triplatin tetranitrate, etc. The alkylating drugs are capable of causing the destruction of cancer cells by binding to DNA within the cancer cells and damaging the DNA structure.

Examples of such antimetabolites include, but are not limited to, pyrimidine derivatives (e.g., 5-fluorouracil, capecitabine, cis-tarabine, gemcitabine, fludarabine, etc.), folate derivatives (e.g., methotrexate, pemetrexed, etc.), and purine derivatives (e.g., mercaptopurine, etc.). Antimetabolites can induce cancer cell death by inhibiting metabolism necessary for DNA replication and cell survival.

Examples of such natural anticancer drugs include, but are not limited to, topoisomerase inhibitors (e.g., camptothecin, epifodophyllotoxin, taxane-based drugs (docetaxel, paclitaxel)), antibiotics (e.g., dactinomycin, doxorubicin, daunorubicin, mitomycin, pleomycin, idarubicin, mitoxantrone HCl, etc.

In the present invention, a targeted anticancer drug is an anticancer drug that induces the death of cancer cells by inhibiting a target protein (receptor or enzyme) involved in the growth of cancer, the meaning of which is as commonly used in the art to which the present invention belongs. The cytotoxic anticancer drugs include target protein (tyrosine kinase, etc.) inhibiting-small molecule compounds and monoclonal antibodies.

In one example, the targeted anticancer drug may be an antibody or a tyrosine kinase inhibitor directed against one or more targets selected from the group consisting of VEGF-A, HER2, and EGFR.

In one embodiment, a targeted anticancer drug that can be co-administered with a malate metal salt is a VEGF-A inhibitor. In the present invention, the VEGF-A inhibitor is a monoclonal antibody such as bevacizumab, ranibizumab, aflibercept, ramucirumab, small molecule compounds such as, but not limited to, sunitinib, pazopanib, sorafenib, and axitinib.

In one embodiment, the targeted anticancer drug that can be co-administered with a malate metal salt is an EGFR inhibitor. In the present invention, the EGFR inhibitors include osimertinib, gefitinib, erlotinib, afatinib, brigatinib, and icotinib, small molecule compounds such as vandetanib, and monoclonal antibodies such as cetuximab, panitumumab, zalutumumab, nimotuzumab, and matuzumab, including, but not limited to, cetuximab, panitumumab, panitumumab, zalutumumab, and matuzumab.

In one embodiment, a targeted anticancer drug that may be co-administered with a malate metal salt may be a HER2 inhibitor, such as, but not limited to, trastuzumab, pertuzumab, lapatinib, neratinib, or afatinib.

Besides, the targeted anticancer drugs of the present invention can also be used in the treatment of Bcr-Abl targeted anticancer drugs, such as imatinib, dasatinib, nilotinib; Src targeted anticancer drugs, such as bosutinib; JAK targeted anticancer drugs, such as lestaurtinib, ruxolitinib, pacritinib; MAP2 kinase-targeting drugs, such as cobimethinib, selumetinib, trametinib, and binimetinib, and MEL4-ALK-targeting drugs, such as ceritibin and crizotinib, without limitation.

The second anticancer drug of the present invention may be a combination of one or more targeted anticancer drugs or immuno-anticancer drugs, which may be administered simultaneously or at different times. In one specific embodiment of the present invention, malate metal salts, or malate metal salts and DCA, in combination with the second anticancer drugs such as sorafenib, trastuzumab (Herceptin), or osimertinib, were treated in several kinds of cancers so that it was found that cancer cells significantly were killed compared to the treatment with the second anticancer drug or malate metal salts (or addition of DCA) alone (See FIG. 7).

In one embodiment, the second anticancer drug of the present invention may be an immuno-oncology drug such as a CAR-T, CAR-NK, immune checkpoint inhibitor, or anticancer vaccine, but not limited to. Immune cells are subjected to energy depletion by the rapid glycolysis of cancer cells in the hypoxic environment within tumor tissue, which is one of the important causes of suppressed immune activity in the context of anticancer therapy. In one specific embodiment of the present invention, it was found that when malate metal salts in combination with IL-2 or IL-12 were treated to T cells and NK cells, the activity of immune cells was significantly enhanced compared to the treatment with cytokines alone (See FIG. 9). Therefore, malate metal salts may have synergistic effects with immuno-oncology drugs such as CAR-T, CAR-NK, immune checkpoint inhibitors, and anticancer vaccines as the second anticancer drug.

Furthermore, the second anticancer drug of the present invention can be selected according to the type and progression of cancer in order to cure, control, or alleviate symptoms of cancer. Since the composition comprising the malate metal salt of the present invention can be used as an anticancer adjuvant by promoting the depletion of NAD⁺ in cancer cells to enhance their responsiveness to the second anticancer drug, the type of second anticancer drug used or the type of cancer is not particularly limited. For example, the cancer may be, but is not limited to, liver cancer, lung cancer, colon cancer, brain cancer, kidney cancer, pancreatic cancer, breast cancer, and cancerous diabetes.

The compositions comprising the malate metal salt of the present invention can be co-administered with the second anticancer drug in a form that is independent of the second anticancer drug, as well as administered in a state of physical/chemical bonding with the second anticancer drug by any method known in the art depending on the purpose. For example, the composition comprising the malate metal salt may be used directly bound to the second anticancer drug, or may be linked to the second anticancer drug via a well-known linker, and are not particularly limited in their method of application as long as the composition comprising the malate metal salt of the present invention acts in combination with the second anticancer drug to produce an enhanced anticancer effect.

Another aspect of the present invention is an anticancer adjuvant pharmaceutical composition comprising the malate metal salt as an active ingredient to enhance a response to an anticancer radiotherapy.

The malate metal salt is as described above.

As used herein, the term “anticancer radiotherapy” refers to a therapeutic practice of irradiating cancer cells or tumor tissue with radiation for the purpose of killing cancer cells. It is generally a standard treatment for controlling inoperable or non-resectable tumors, or tumor metastases, and is based on the principle that radiation delivered to a targeted area causes the death of reproductive cells. In the present invention, the anticancer radiotherapy may be, but is not limited to, an ionizing radiation therapy, an electromagnetic radiation, a brachytherapy, or an external beam radiation therapy.

The composition comprising the malate metal salt according to the present invention exhibit synergistic anticancer effects when combined with a radiation therapy and thus may be useful as an anticancer adjuvant to the radiation therapy or as a radio-sensitizer to enhance radio-sensitivity, and the types of cancers to which they may be applied are not particularly limited. For example, the cancers may be, but are not limited to, liver cancer, lung cancer, colon cancer, brain cancer, kidney cancer, pancreatic cancer, breast cancer, and cancerous diabetes.

The invention may be practiced in many other forms, and the scope of the invention is not limited to the embodiments described hereinafter. Furthermore, the embodiments are provided for the purpose of more fully explaining the invention to one of ordinary skill in the art. Furthermore, throughout the specification, references to “including” any component are not intended to exclude other components, but rather to allow for the inclusion of additional components, unless specifically noted to the contrary.

### ADVANTAGEOUS EFFECTS

The composition for preventing or treating cancer comprising the malate metal salt of the present invention as the active ingredient have excellent anticancer effects by depleting NAD⁺ being an energy source in cancer cells and increasing immune activity, and increasing insulin secretion by increasing malic acid activity, which can solve problems such as decreased insulin secretion or resistance induced by toxicity caused by various anticancer treatments. In addition, when administered in combination with other anticancer drugs or radiotherapy, it has excellent effect as an anticancer adjuvant by enhancing the response to anticancer drugs and radiotherapy.

### BRIEF DESCRIPTION OF THE DRAWINGS

FIG. 1 is a graph comparing the levels of MDH2 with ME2 and ME3 when cancer cells are treated with CaMal, CaMal₂, or CaMal-DCA.

FIG. 2 is a graph comparing NAD⁺ levels when cancer cells are treated with CaMal, CaMal₂, or CaMal-DCA.

FIG. 3 is a graph comparing insulin secretion levels according to the presence and absence of glucose in pancreatic beta cells treated with CaMal or CaMal₂.

FIG. 4 is a graph comparing a survival rate of cancer cells according to CaMal treatment concentration.

FIG. 5 is a graph comparing a survival rate of cancer cells according to CaMal₂ treatment concentration.

FIG. 6 is a graph comparing a survival rate of cancer cells according to CaMal-DCA treatment concentration.

FIG. 7 is a graph comparing a survival rate of cancer cells administered with CaMal and CaMal₂, or CaMal-DCA alone or in combination with a second anticancer drug.

FIG. 8 is a graph comparing a survival of cancer cells treated with CaMal and CaMal₂, or CaMal-DCA and radiation alone or in combination.

FIG. 9 is a graph comparing the levels of activated T and NK cells treated with IL-2 alone or in combination with IL-2 or IL-12 and CaMal₂.

FIG. 10 is a graph showing a tumor growth inhibition effect of subcutaneous administration of CaMal of pancreatic cancer xenograft mice.

FIG. 11 is a graph showing a tumor growth inhibition effect of oral administration of CaMal₂ of in liver cancer xenograft mice.

FIG. 12 is a graph showing a tumor growth inhibition effect of intravenous administration of CaMal-DCA of mammary cancer xenograft mice.

### BEST MODE FOR CARRYING OUT THE INVENTION

Hereinafter, embodiments of the present disclosure will be described in detail with reference to the accompanying drawings so that those skilled in the art to which the present disclosure pertains may easily carry out the invention. However, the present disclosure may be embodied in many different forms and is not limited to the embodiments described herein. Throughout the specification, the same reference numerals are used for similar parts.

### EXPERIMENTAL EXAMPLE 1

### Confirmation of Metabolic Regulatory Effects of Malate Metal Salts in Cancer Cells

### 1-1. Changes in the Levels of MDH2 (Malate Dehydrogenase 2), ME2 (Malic Enzyme 2) and Me3 (Malic Enzyme 3)

1×10⁶ liver cancer (HepG2), breast cancer (MDA-MB-231), or lung cancer (H1975) cells were treated with calcium malate (CaMal) of 5 mM, calcium dimalate (CaMal₂) of 2.5 mM, or calcium malate-dichloroacetic acid (CaMal-DCA) of 1 mM for 48 hours, respectively. After that, proteins were then extracted using RIPA buffer. Biorbyt's Malate dehydrogenase 2 (MDH2), Malic enzyme 2 (ME2) and Malic enzyme 3 (ME3) ELISA assay kits were utilized to quantitatively analyze the respective factors in the above extracted proteins.

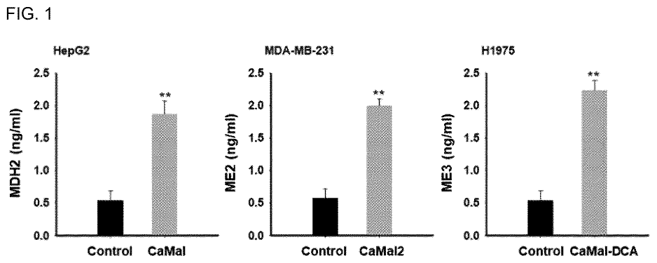

The treatment of the liver cancer cells, breast cancer cells, and lung cancer cells with CaMal, CaMal₂, or CaMal-DCA, respectively, resulted in a significant increase in the amounts of MDH2, ME2, and ME3 compared to untreated controls (See FIG. 1; **p<0.001).

### 1-2. Changes in NAD⁺ Levels

1×10⁶ liver cancer (HepG2), breast cancer (MDA-MB-231), or lung cancer (H 1975) cells were treated with CaMal of 5 mM, CaMal₂ of 2.5 mM, or CaMal-DCA of 1 mM for 48 hours, respectively. Then, NAD⁺ levels were then analyzed according to the method of Abcam's NAD⁺ assay kit.

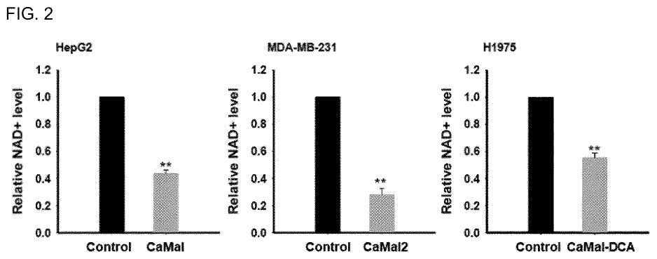

The treatment of the liver cancer cells, breast cancer cells, and lung cancer cells with CaMal, CaMal₂, or CaMal-DCA, respectively, resulted in a significant decrease in the levels of NAD⁺ in all compared to untreated controls (See FIG. 2; **p<0.001).

### 1-3. Changes in Insulin Levels

1×10⁶ beta cells were treated with CaMal of 5 mM or CaMal₂ of 2.5 mM for 48 hours in the absence or presence of glucose of 5 mM, respectively, and a culture medium was harvested. The medium was placed in a spin concentrator (Corning, Inc.) and run at 7,500 g for 10 minutes to obtain insulin assay samples. The samples were quantitatively analyzed using an assay kit manufactured by Enzo company.

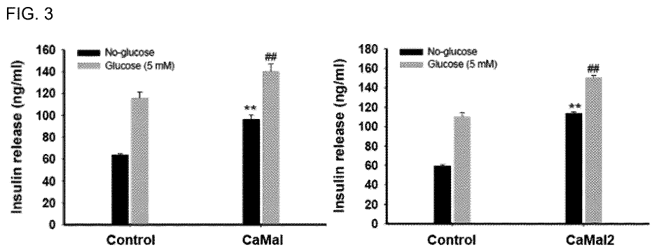

The treatment of the beta cells with CaMal or CaMal₂ resulted in a significant increase in insulin levels compared to untreated controls in both the absence and presence of glucose (See FIG. 3; **p<0.001 vs. Control (No-glucose); ##p<0.001 vs Control (Glucose)).

### EXPERIMENTAL EXAMPLE 2

### Anticancer Effects of Malate Metal Salts in Vitro

### 2-1. Anticancer Effect of CaMal

3×10³ colon cancer (HCT-116) or brain cancer (U-87MG) cells were incubated for 24 hours and then treated with CaMal of 0.5, 1, 2.5, or 5 mM and incubated for another 96 hours. Then, 3-(4,5-dimethylthiazol-2-yl)-2,5-diphenyltetrazolium bromide reagent of 10 μl was added to each well and reacted for 1 hour. After removing the reagent at the end of the reaction, DMSO of 200 μl was added to each well and then the absorbance was measured to confirm a survival rate of the cells.

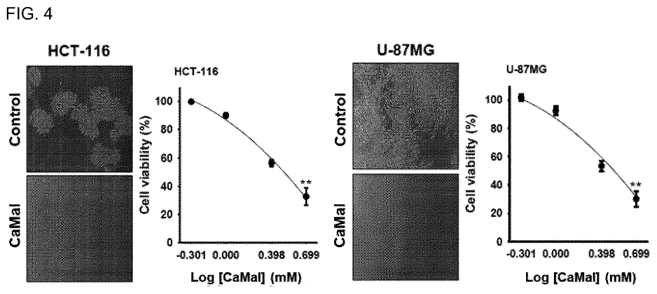

The treatment of the colon cancer (HCT-116) or brain cancer (U-87MG) cells with CaMal resulted in a concentration-dependent manner decrease in a survival rate of the cancer cells, which was significant (See FIG. 4; **p<0.001 vs 0.5, 1, and 2.5 mM groups).

### 2-2. Anticancer Effect of CaMal 2

3×10³ kidney cancer (Caki-1) or pancreatic cancer (Aspc-1) cells were incubated for 24 hours and then treated with CaMal₂ of 0.5, 1, 2.5, or 5 mM and incubated for 96 hours again. Then, 3-(4,5-dimethylthiazol-2-yl)-2,5-diphenyltetrazolium bromide reagent of 10 μl was added to each well and reacted for 1 hour. After removing the reagent at the end of the reaction, DMSO 200 of μl was added to each well and the absorbance was measured to confirm the survival rate of the cells.

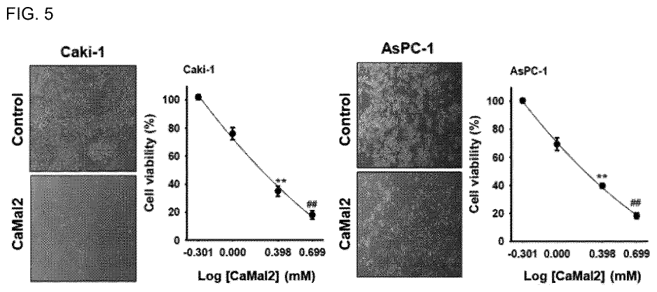

The treatment of the kidney (Caki-1) or pancreatic (Aspc-1) cancer cells with CaMal₂ decreased the survival rate of the cancer cells in a concentration-dependent manner, and these results were found to be significant (See FIG. 5; **p<0.001 vs 0.5, 1 mM groups. ##p<0.001 vs 0.5, 1, 2.5 mM groups).

### 2-3. Anticancer Effect of CaMal-DCA

3×10³ liver cancer (HepG2), lung cancer (H1975), pancreatic cancer (Aspc-1), or breast cancer (MDA-MB-231) cells were cultured for 24 hours and then treated with CaMal-DCA of 0.5, 1, 2.5, or 5 mM and incubated for another 96 hours. Then, 3-(4,5-dimethylthiazol-2-yl)-2,5-diphenyltetrazolium bromide reagent of 10 μl was added to each well and reacted for 1 hour. After removing the reagent at the end of the reaction, DMSO of 200 μl was added to each well and the absorbance was measured to confirm the survival rate of the cells.

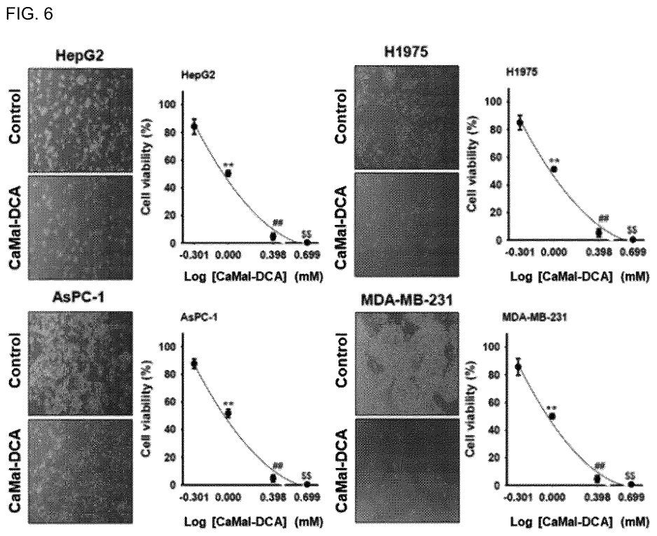

The treatment of the liver cancer (HepG2), lung cancer (H1975), pancreatic cancer (Aspc-1), or breast cancer (MDA-MB-231) cells with CaMal-DCA decreased the survival rate of the cancer cells in a concentration-dependent manner, and these results were significant (See FIG. 6; **p<0.001 vs 0.5 groups, ##p<0.001 vs 0.5, 1 mM groups; $$p <0.001 vs 0.5, 1, 2.5 mM groups).

### EXPERIMENTAL EXAMPLE 3

### Anticancer Effect of Combining Anticancer Drugs with Malate Metal Salts

3×10³ liver cancer (HepG2), breast cancer (MDA-MB-231) or lung cancer (H1975) cells were cultured for 24 hours. Then, the liver cancer cells were treated with sorafenib of 1 μM and CaMal of 5 mM alone or in combination, the breast cancer cells were treated with Herceptin of 2.5 μM and CaMal₂ of 2.5 mM alone or in combination, and the lung cancer cells were treated with Osimertinib of 1 nM and CaMal-DCA of 1 mM alone or in combination and incubated for another 96 hours. After that, 3-(4,5-dimethylthiazol-2-yl)-2,5-diphenyltetrazolium bromide reagent of 10 μl was added to each well and reacted for 1 hour. After removing the reagent at the end of the reaction, DMSO of 200 μl was added to each well and the absorbance was measured to confirm the survival rate of the cells.

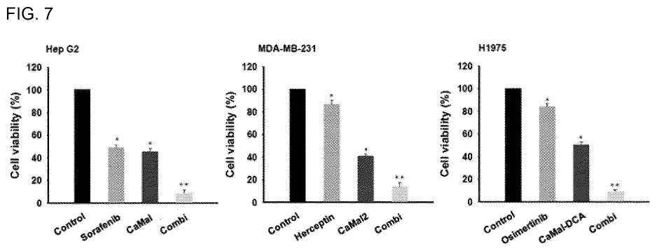

When the liver cancer (HepG2), breast cancer (MDA-MB-231), or lung cancer (H1975) cells were treated with malate metal salts and anticancer drugs alone or in combination, the malate metal salts were found to be as effective as or more effective than conventional anticancer drugs known to have good anticancer effects even when administered alone. In particular, when each of anticancer drugs and malate metal salts were treated in combination, they exhibited synergistic effects so that it was found that all cancer cells were almost killed. These results suggest that co-administration of malate metal salts with anticancer drugs can overcome anticancer drug resistance and show excellent anticancer effects (See FIG. 7; *p<0.001 vs Control, **p<0.001 vs. Control, single treatment groups).

### EXPERIMENTAL EXAMPLE 4

### Anticancer Effect of Combining Radiation and Malate Metal Salts

3×10³ liver cancer (HepG2), breast cancer (MDA-MB-231), or lung cancer (H1975) cells were cultured for 24 hours. Then, each of cells was then treated with radiation of 2 Gy dose and CaMal of 5 mM, CaMal₂ of 2.5 Mm, or CaMal-DCA of 1 mM, alone or in combination, for 48 hours. Then, 3-(4,5-dimethylthiazol-2-yl)-2,5-diphenyltetrazolium bromide reagent of 10 μl was added to each well and reacted for 1 hour. After removing the reagent at the end of the reaction, DMSO of 200 μl was added to each well and the absorbance was measured to confirm the survival rate of the cells.

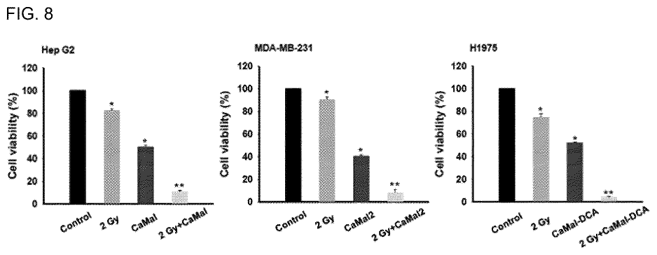

The treatment of the liver cancer (HepG2), breast cancer (MDA-MB-231), or lung cancer (H1975) cells with malate metal salts and radiation alone or in combination showed that the malate metal salts killed cancer cells effectively rather than irradiation of 2Gy. When the malate metal salts were treated in combination with radiation, it was shown that an anticancer efficacy was enhanced. These results suggest that the resistance to irradiation can be overcome by combining the malate metal salts with irradiation (See FIG. 8; *p<0.001 vs. Control, **p<0.001 vs. Control, single treatment groups).

### EXPERIMENTAL EXAMPLE 5

### Immune Cell Activation Effect of Malate Metal Salts

T cells were isolated from spleen and lymphoid tissues of C57bl/6 mice utilizing a mouse CD8 isolation kit (ThermoFisher). These T cells were treated with 20 units of IL-2 alone or in combination with CaMal₂ of 2.5 mM and then CD25⁺ and CD69⁺ cells were separated by FACS to quantify activated T cells.

Next, NK cells were isolated from the spleens of C57bl/6 mice utilizing a mouse NK cell isolation kit (ThermoFisher). The above NK cells were treated with IL-12 of 2 ng/ml alone or in combination with CaMal₂ of 2.5 mM, and CD16⁻ and CD56⁺ cells were sorted by FACS to quantify activated NK cells.

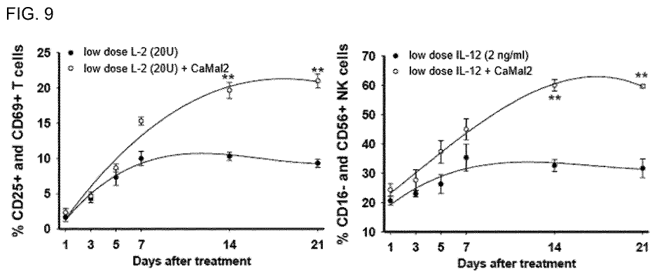

As a result, it was confirmed that the number of activated T cells significantly increased in a group where the IL-2 and malate metal salts were treated at the same time compared to a group where the IL-2 was treated alone (See left in FIG. 9), and the number of activated NK cells significantly increased in a group where the IL-2 and malate metal salts were treated at the same time compared to a group where the IL-2 was treated alone (See right in FIG. 9; **p<0.001 vs low dose IL-2 or IL-12 group).

### EXPERIMENTAL EXAMPLE 6

### Anticancer Effect of Malate Metal Salts in Vivo

### 6-1. Anticancer Effect of CaMal

1×10⁷ pancreatic cancer (Aspc-1) cells were implanted with Athymic Balb/c nude mice and when they grew to approximately 200 mm³, CaMal of 20 mg/kg was administered subcutaneously twice a week to monitor tumor growth for 30 days.

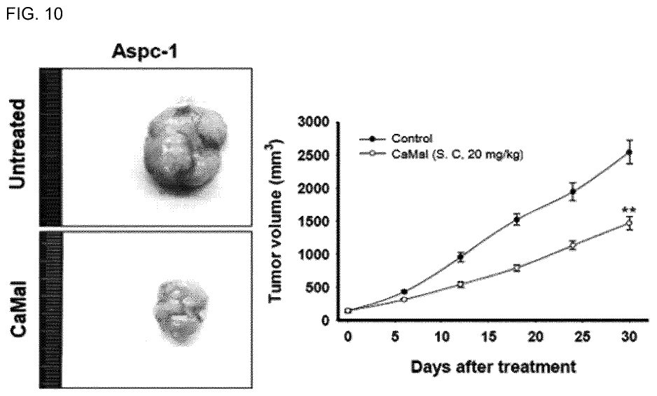

The CaMal was administered to the pancreatic cancer (Aspc-1) cell-transplanted mice so that it was confirmed that the tumor size became reduced visually and clearly compared to untreated controls (See left in FIG. 10), and these results were confirmed at a statistically significant level (See right in FIG. 10; **p<0.001 vs. Control).

### 6-2. Anticancer Effect of CaMal 2

1×10⁷ liver cancer (HepG2) cells were implanted with Athymic Balb/c nude mice and when they grew to approximately 200 mm³, CaMal₂ of 80 mg/kg was administered orally five times a week to observe tumor growth for 30 days.

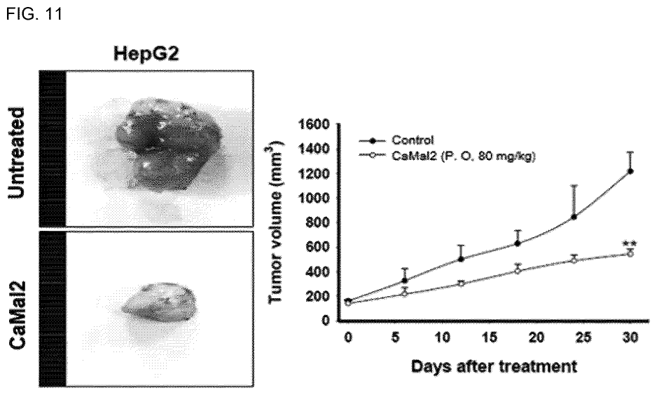

The CaMal₂ was treated to liver cancer (HepG2) cell-transplanted mice so that it was confirmed that tumor size became reduced visually and clearly compared to untreated controls (See left in FIG. 11), and these results were confirmed at a statistically significant level (See right in FIG. 11; **p<0.001 vs. Control).

### 6-3. Anticancer Effect of CaMal-DCA

1×10⁷ breast cancer (MDA-MB-231) cells were implanted with Athymic Balb/c nude mice and when they grew to approximately 200 mm³, CaMal-DCA of 10 mg/kg was administered intravenously once a week to monitor tumor growth for 30 days.

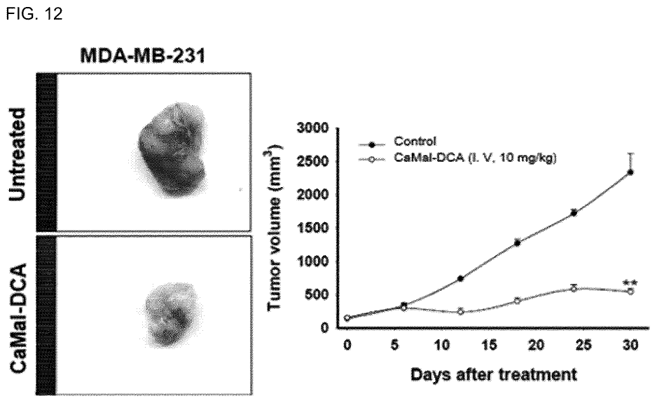

The CaMal-DCA was treated to breast cancer (MDA-MB-231) cell-transplanted mice so that it was confirmed that tumor size became reduced visually and clearly compared to untreated controls (See left in FIG. 12), and these results were confirmed at a statistically significant level (See right in FIG. 12; **p<0.001 vs. Control).

From the above description, it will be understood by those skilled in the art to which the invention belongs that the invention may be practiced in other specific forms without altering its technical idea or essential features. In this regard, it should be understood that the embodiments described above are exemplary in all respects and are not intended to be limiting. The scope of the invention is to be construed as including the meaning and scope of the patent claims that follow, and all modifications or variations derived from the equivalent concepts thereof, rather than the detailed description above.

1. A pharmaceutical composition for preventing or treating cancer comprising a malate metal salt as an active ingredient.

2. The pharmaceutical composition of claim 1, wherein the malate metal salt is calcium malate or calcium di-malate.

3. The pharmaceutical composition of claim 1, wherein the cancer is at least one selected from the group consisting of liver cancer, lung cancer, colorectal cancer, brain cancer, kidney cancer, pancreatic cancer, breast cancer and cancerous diabetes.

4. The pharmaceutical composition of claim 1, wherein the pharmaceutical composition further comprises DCA (Dichloroacetic acid).

5. The pharmaceutical composition of claim 1, wherein the pharmaceutical composition is administered subcutaneously, orally, or intravenously.

6. An anticancer adjuvant pharmaceutical composition comprising a malate metal salt as an active ingredient for enhancing a response to a second cancer drug.

7. The anticancer adjuvant pharmaceutical composition of claim 6, wherein the malate metal salt is calcium malate or calcium di-malate.

8. The anticancer adjuvant pharmaceutical composition of claim 6, wherein the second cancer drug is a targeted anticancer drug or an immune anticancer drug.

9. The anticancer adjuvant pharmaceutical composition of claim 6, wherein a cancer is targeted by the second cancer drug is at least one selected from the group consisting of liver cancer, lung cancer, colorectal cancer, brain cancer, kidney cancer, pancreatic cancer, breast cancer and cancerous diabetes.

10. A pharmaceutical composition for preventing or treating cancer comprising a malate metal salt and a second cancer drug as an active ingredient.

11. An anticancer adjuvant pharmaceutical composition comprising a malate metal salt as an active ingredient for enhancing a response to an anticancer radiotherapy.

12. The anticancer adjuvant pharmaceutical composition of claim 11, wherein the malate metal salt is calcium malate or calcium di-malate.

13. The anticancer adjuvant pharmaceutical composition of claim 11, wherein a cancer is targeted by the anticancer radiotherapy is at least one selected from the group consisting of liver cancer, lung cancer, colorectal cancer, brain cancer, kidney cancer, pancreatic cancer, breast cancer and cancerous diabetes.
